(19) United States
(12) Patent Application Publication
(10) Pub. No.: US 2023/0256251 A1
(43) Pub. Date: Aug. 17, 2023

(54) DETECTION OF UNINTENTIONAL AND INTENTIONAL BODY SIGNALS TO CONTROL DEVICE STIMULATION

(71) Applicant: Medtronic, Inc., Minneapolis, MN (US)

(72) Inventors: Sarah J. OFFUTT, Golden Valley, MN (US); Leonid M. LITVAK, Los Angeles, CA (US); Maneesh SHRIVASTAV, Blaine, MN (US)

(21) Appl. No.: 18/104,880

(22) Filed: Feb. 2, 2023

Related U.S. Application Data
(60) Provisional application No. 63/305,885, filed on Feb. 2, 2022.

(51) Int. Cl. A61N 1/36; A61N 1/372; A61N 1/05

(52) U.S. Cl. CPC A61N 1/36139; A61N 1/37211; A61N 1/0551

(57) ABSTRACT

An implantable tibial nerve electrical stimulation therapy device, system and method configured to detect unintentional and intentional body signals to control and modify the electrical stimulation therapy, thereby enabling selective pausing of electrical stimulation therapy and increase/decrease in amplitude or frequency of the electrical stimulation therapy for improved safety, comfort and effective therapy.

### CROSS-REFERENCE TO RELATED APPLICATIONS

This application claims the benefit of U.S. Provisional Application Ser. No. 63/305,885, filed Feb. 2, 2022, the disclosure of which is hereby incorporated by reference herein.

### FIELD

The present technology is generally related to methods, systems and devices related to electrical stimulation therapy and more particularly to detection of unintentional and intentional body signals to control electrical stimulation therapy.

### BACKGROUND

Overactive bladder syndrome is a condition where there is a frequent feeling of the need to urinate to a degree that it negatively affects a person's life. Overactive bladder syndrome is characterized by a group of four symptoms: urgency (e.g., a sudden, compelling desire to pass urine that is difficult to defer), urinary frequency (e.g., feeling the need to urinate more than eight times per day), nocturia (e.g., interrupted sleep caused by an urge to urinate), and urge incontinence (urinary incontinence characterized by the involuntary loss of urine occurring for no apparent reason while urinary urgency is experienced). As many as 30% of men and 40% of women in the United States live with overactive bladder syndrome; the economic cost associated with this disorder is estimated to be in the billions of dollars each year.

Unfortunately, it is often difficult to successfully treat overactive bladder syndrome through traditional behavioral methods, such as pelvic floor exercises and bladder training. Medications are typically no more effective than behavioral methods, and are often associated with negative side effects. More recently, various methods of electrical nerve stimulation have been developed. One promising treatment method includes tibial nerve stimulation therapy.

Tibial nerve stimulation therapy is a treatment that uses a small device to send mild electrical impulses to the tibial nerves (e.g., acupoints SP6, KI7, and/or KI8), which can generally be accessed at a shallow depth within the lower leg of a patient. The tibial nerves influence the behavior of structures such as the bladder, sphincter and pelvic floor muscles. In some cases, electrical stimulation of the tibial nerves can successfully eliminate or reduce overactive bladder syndrome, as well as a host of other bodily disorders including urinary incontinence, urinary urge/frequency, urinary retention, pelvic pain, fecal incontinence, bowel dysfunction (constipation, diarrhea, etc.), neurogenic bladder, and sexual dysfunction among others.

Conventional electrical nerve stimulation (defined at a high level by stimulation pulse training characterized by amplitude, pulse width and rate), provided by implantable neurostimulation devices or neuromodulation devices is typically delivered continuously (e.g., without interruption). However, with tibial nerve stimulation, flexion of the lower foot of a patient can cause changes in depth of the tibial nerve relative to the therapy delivering electrodes. Accordingly, depending upon the position of the lower foot, electrical stimulation therapy may be uncomfortable in some positions and below the prescribed therapeutic level in other positions. Moreover, stimulation therapy during particular activities, such as operating heavy machinery or driving a vehicle, may be hazardous. The present disclosure addresses these concerns.

### SUMMARY

The techniques of this disclosure generally relate to a tibial nerve electrical stimulation therapy device, system and method configured to detect unintentional and intentional body signals to control and modify the electrical stimulation therapy. To enable flexibility in treatment during peripheral nerve stimulation, devices of the present disclosure are configured to adapt to changes of daily living to sense when it is safe to activate stimulation, as well as to sense changes in the depth of the targeted nerve, dependent upon flexion of a limb of a patient. The flexion of the limb may change the configuration of the implant from its original position, or may change the location of the targeted nerve with respect to the implant. Some of the techniques described in this disclosure compensate for the change in distance of the targeted nerve from the therapy delivering electrode due to such flexion. The movement of the foot could include dorsiflexion, plantar flexion, inversion, and eversion as any one of these or a combination of such movements has potential to move the original electrode placement, or wireless stimulation boundary, or change the location of the targeted nerve with respect to the implant. Embodiments of the present disclosure can include a sensor (e.g., accelerometer, microphone, EMG sensor, etc.) configured to detect patient motion and other activity (e.g., driving, etc.) as an input for determining whether it is safe to activate stimulation. If stimulation is to occur during periods of movement, the amplitude of stimulation can be adjusted to match the depth of the targeted nerve (e.g., tibial nerve) as the patient moves their limb (e.g., lower foot) through a normal range of motion in order to avoid discomfort or delivery of therapy above or below a threshold that can provide a suitable, sustainable, and safe therapeutic benefit (e.g., when the targeted nerve is in close proximity to the implantable electrical stimulation device) and to improve patient outcomes by providing a more consistent electrical stimulation therapy.

An additional embodiment could include a leadless electrode system with an intended stimulation area. Additionally, a separate embodiment could include a leaded system with ring, cuff, paddle, or spiral electrodes arranged on a lead.

Further, in some embodiments, the device, system and method can use the same sensor or sensors to receive user commands (e.g., user-defined tactile command, etc.) to turn on/off stimulation therapy, skip a stimulation therapy session, or change the amplitude of the stimulation therapy. For example, user defined commands such as tapping on the skin of the patient in proximity to the implantable electrical stimulation device, cupping the implant area with a palm, moving the leg of the patient in a circle, pointing the toe of the patient, etc., can be sufficient to affect modification of the stimulation therapy. Further, in some embodiments, one or more user-defined commands can enable on-demand stimulation outside of a regularly scheduled therapy regimen to meet the needs of the patient.

Accordingly, embodiments of the present disclosure use body movement to inform stimulation control of the implantable electrical stimulation device. Sensors in the device can detect activity of daily living or directed motion to turn stimulation on/off, up/down, or skip a session, thereby improving safety (e.g., not providing electrical stimulation while operating heavy machinery, avoiding patient discomfort, etc.) with ease (e.g., no external communications or complex instructions required), while optimizing therapy for improved results.

One embodiment of the present disclosure provides an implantable neurostimulation device, including circuitry for generating neurostimulation therapy pulses, one or more electrodes configured to deliver the neurostimulation therapy pulses to a patient, and at least one sensor configured to detect at least one signal, wherein in the detected at least one signal is used by the circuitry to modify the electrical stimulation therapy.

In one embodiment, the implantable neurostimulation device is adapted for tibial nerve stimulation. In one embodiment, the one or more sensor is at least one of an accelerometer, microphone, or electromyographic sensor. In one embodiment, the one or more signal is a user defined command detectable by the one or more sensor within the implantable neural stimulation device. In one embodiment, the at least one signal correlates to patient operation of at least one of vehicle (automobile, car, etc.) or heavy machinery with a means to easily bypass this functionality. In one embodiment, the implantable neurostimulation device is configured to selectively pause or suspend the electrical stimulation therapy. In one embodiment, at least one signal correlates to a distance between a targeted nerve and the one or more electrodes. In one embodiment, the implantable neurostimulation device is configured to selectively increase or decrease at least one of an amplitude or frequency or pulse width or phase of the electrical stimulation therapy based on a determined distance between a targeted nerve and the implantable neural stimulation device. In one embodiment, the implantable neurostimulation device can be configured to communicate with at least one of a mobile computing device, smart wristwatch, a desktop computer or a dedicated implantable neurostimulation device programmer.

Another embodiment of the present disclosure provides an implantable neurostimulation system including an implantable neurostimulation device configured to deliver electrical energy via one or more electrodes to a patient according to a prescribed dosing pattern for the treatment of one or more physiological conditions, and an external sensor configured to detect at least one signal, wherein the at least one signal detected by the external sensor is wirelessly communicated to the implantable neurostimulation device to modify the electrical stimulation therapy.

In one embodiment, the implantable neurostimulation device is adapted for tibial nerve stimulation. In one embodiment, the external sensor is at least one or more of a heart rate monitor, pulse oximeter, glucose monitor, respiratory sensor, perspiration sensor, posture orientation sensor, motion sensor, accelerometer, or microphone. This embodiment can be multitude or conglomeration of several such sensors. In one embodiment, the at least one signal correlates to patient operation of at least one of an automobile or heavy machinery. In one embodiment, the implantable neurostimulation device is configured to selectively pause or suspend the prescribed dosing pattern. In one embodiment, the at least one signal correlates to a distance between a targeted nerve and the one or more electrodes. In one embodiment, the implantable neurostimulation device is configured to selectively increase or decrease at least one of an amplitude, pulse width, shape (square, sinusoidal, or triangular) or frequency or phase of the prescribed dosing pattern based on a determined distance between a targeted nerve and the implantable neural stimulation device. In one embodiment, the implantable neurostimulation device is configured to communicate with at least one of a mobile computing device, smart wristwatch, a desktop computer or a dedicated implantable neurostimulation device programmer.

Another embodiment of the present disclosure provides an implantable neurostimulation system including an implantable neurostimulation device configured to deliver electrical energy via one or more electrodes to a patient according to a prescribed dosing pattern for the treatment of one or more physiological conditions, the implantable neural stimulation device including one or more sensor configured to detect at least one signal, wherein in the detected at least one signal is used to modify the prescribed dosing pattern, and an external programmer configured to wirelessly communicate with the implantable neurostimulation device.

In one embodiment, the implantable neurostimulation device is adapted for tibial nerve stimulation. In one embodiment, the external programmer comprises an external sensor configured to detect at least one signal, wherein the at least one signal detected by the external sensor is wirelessly communicated to the implantable neurostimulation device to modify the prescribed dosing pattern. In one embodiment, the implantable neurostimulation device is configured to at least one of selectively pause the prescribed dosing pattern or selectively increase or decrease at least one of an amplitude or pulse width or frequency of the prescribed dosing pattern.

The details of one or more aspects of the disclosure are set forth in the accompanying drawings and the description below. Other features, objects, and advantages of the techniques described in this disclosure will be apparent from the description in the drawings, and from the claims.

### BRIEF DESCRIPTION OF DRAWINGS

The disclosure can be more completely understood in consideration of the following detailed description of various embodiments of the disclosure, in connection with the accompanying drawings, in which:

FIG. 1 is a schematic diagram depicting a medical system including an implantable device configured to detect unintentional and intentional body signals to control and modify electrical stimulation therapy, in accordance with an embodiment of the disclosure.

FIG. 2 is an exploded, perspective view depicting an implantable neurostimulation device configured to detect unintentional and intentional body signals to control and modify electrical stimulation therapy, in accordance with an embodiment of the disclosure.

FIG. 3 is a block diagram depicting an implantable neural stimulation device and programmer configured to detect unintentional and intentional body signals to control and modify electrical stimulation therapy, in accordance with an embodiment of the disclosure.

FIG. 4A is a graphical representation depicting a partial therapeutic regimen, in accordance with an embodiment of the disclosure.

FIG. 4B is the graphical representation of FIG. 4A, further depicting at least one pause in the therapeutic regimen, in accordance with an embodiment of the disclosure.

FIG. 5 is a graphical representation depicting a therapy regimen in which an amplitude of individual electrical pulses of the therapy regimen are plotted over a period of time, in accordance with an embodiment of the disclosure.

FIG. 6 is a flowchart depicting a method of detecting unintentional and intentional body signals to control and modify delivered electrical stimulation therapy, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

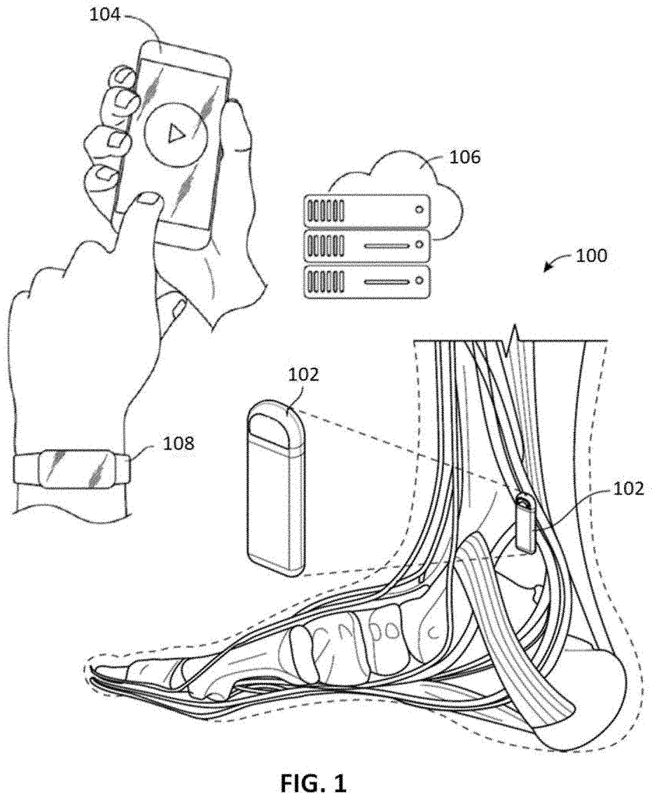

Referring to FIG. 1, a medical system 100 comprising an implantable device 102 configured to detect unintentional and intentional body signals to control and modify electrical stimulation therapy, is depicted in accordance with an embodiment of the disclosure. In embodiments, the neurostimulator device 102 can be an implantable electrical neural stimulation or neuromodulation device, configured to provide electrical stimulation therapy to one or more targeted nerves over an extended period of time. As depicted, the neurostimulator device 102 can be implanted under the skin and cutaneous fat layer via a small incision (e.g., about one to five cm) above the tibial nerve on the medial aspect of the patient's ankle. The neurostimulator device 102 can be positioned adjacent to the region defined by the flexor digitorum longus, flexor halluces longus, and soleus in which the tibial nerve is contained, and implanted adjacent to and proximal to a facia layer. In some embodiments, the neurostimulator device 102 can be a leadless device, in which a primary and a secondary electrode of the neurostimulator device 102 are configured to face towards the tibial nerve upon implantation. In other embodiments, the neurostimulator device 102 can include a lead with a stimulation electrode thereon, configured to be implanted near a tibial nerve. The type of electrode may encompass any one or combination of ring electrodes, cuff like electrodes, spiral stimulation leads, or paddle electrodes that do not directly contact a nerve.

Various example embodiments of neuromodulation or neurostimulation devices and systems are described herein for managing duty cycled electrical nerve stimulation delivered to a subject. Although a specific example of tibial neuromodulation is provided, it is to be appreciated that the concepts disclosed herein are extendable to other types of neurostimulation devices. Further, while the treatment of overactive bladder syndrome is provided as one example therapy regimen, embodiments of the present disclosure can be used to treat a host of other bodily disorders including, but not limited to, urinary incontinence, urinary urge/frequency, urinary retention, pelvic pain, fecal incontinence, bowel dysfunction (constipation, diarrhea, etc.), and sexual dysfunction among others. It is also to be appreciated that the term “clinician” refers to any individual that can prescribe and/or program neuromodulation with any of the example embodiments described herein or alternative combinations thereof. Similarly, the term “patient” or “subject,” as used herein, is to be understood to refer to an individual or object in which the neuromodulation therapy is to occur, whether human, animal, or inanimate. Various descriptions are made herein, for the sake of convenience, with respect to the procedures being performed by a clinician on a patient or subject (the involved parties collectively referred to as a “user” or “users”) while the disclosure is not limited in this respect.

In some embodiments, the medical system 100 can further include an optional external programmer 104 and optional server 106 configured to communicate with the neurostimulator device 102. In some embodiments, the programmer 104 can be a handheld, wireless portable computing device, such as a cellular telephone, tablet, smart wrist watch, dedicated implantable device programmer, or the like. Further, in some embodiments, the medical system 100 can include one or more external or internalized physiological sensors 108, which can be in communication with the neurostimulator device 102, optional external programmer 104, and optional server 106. In one embodiment, one or more physiological sensors 108 can be incorporated into the neurostimulator device 102 or the external programmer 104. In one embodiment, a physiological sensor 108 can be worn by the patient (e.g., a smart watch, wristband tracker, sensors embedded in clothing, etc.), carried by the patient (e.g., a smart phone, mobile computing device, etc.), or positioned in proximity to the patient (e.g., a stationary monitor, etc.). Examples of physiological sensors 108 include a heart rate monitor, pulse oximeter, respiratory sensor, perspiration sensor, posture orientation sensor, motion sensor, accelerometer, microphone, electromyographic (EMG) sensor or the like.

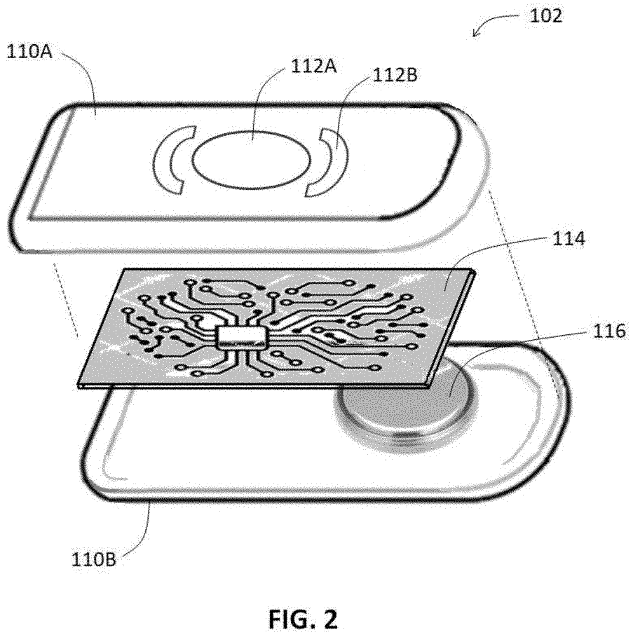

Referring to FIG. 2, an exploded perspective view of an implantable neurostimulation device 102 configured to detect unintentional and intentional body signals to control and modify electrical stimulation therapy, is depicted in accordance with an embodiment of the disclosure. In embodiments, the neurostimulator device 102 can generally include a housing 110A/B, one or more electrodes 112A/B (e.g., anode, cathode, etc.), and an associated computing device 114 and power source 116. The housing 110 can be constructed of a material that is biocompatible and hermetically sealed, such as titanium, tantalum, stainless steel, plastic, ceramic, or the like. In some embodiments, the electrodes 112A/B can be formed integrally as part of, or anchored to, the housing 110, such that the electrodes 112A/B can be positioned directly adjacent to a selected nerve for stimulation or other specified target tissue stimulation site known to moderate or affect a patient's physiological or health condition. The battery of such device could be integrated into the body of the hermetically sealed capsule.

Once the implantable neurostimulator device is anchored at the targeted stimulation site, electrical stimulation can be applied using a low intensity, low frequency and low duty cycle stimulation regimen. Applying electrical stimulation therapy at low intensities, low frequencies and low duty cycles is also a key feature of embodiments of the disclosure, as it enables a power source of the neurostimulator device 102 to be small, yet still with sufficient capacity to uniformly carry out the stimulation protocol or stimulation regimen for several weeks, months or years, thereby reducing the amount of time that a patient has to spend recharging or replacing the power source. For example, in some embodiments, a suitable intensity of the electrical stimulation therapy can be between about 0.1 mA and about 25 mA, although electrical stimulation therapy outside of this range is also contemplated.

The one or more electrodes 112A/B can take on various forms. For example, in some embodiments, at least one of the electrodes 112 can be in the form of a smooth surface electrode, without any sharp or pointed edges. Alternatively, in other embodiments, one or more of the electrodes 112 may be located at the distal and of a short lead, which can generally be between about 10 mm and about 50 mm in length, sometimes referred to as a “pigtail lead,” as it can be attached to one end of the housing 110.

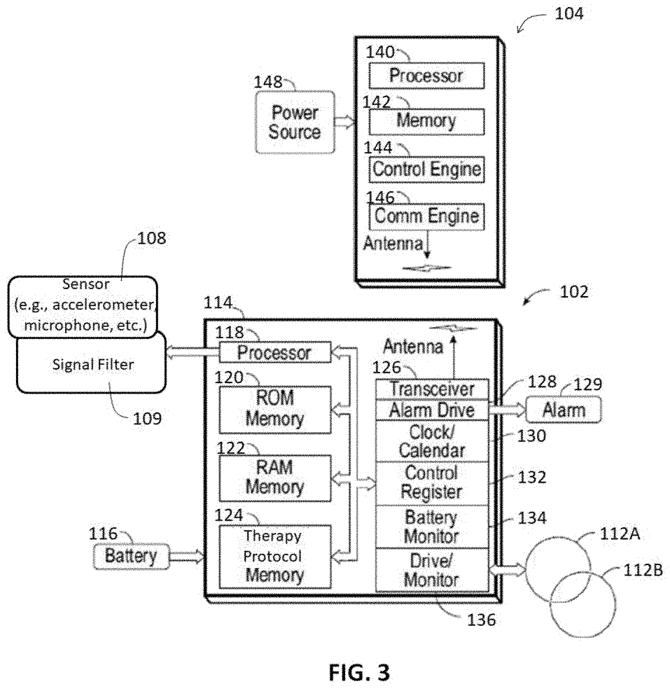

Referring to FIG. 3, a block diagram of an implantable neurostimulator device 102 and programmer 104 configured to detect unintentional and intentional body signals to control and modify electrical stimulation therapy, is depicted in accordance with an embodiment of the disclosure. The implantable neurostimulator device 102 can include a computing device 114, which can be carried in the housing 110 (as depicted in FIG. 2) and can be in electrical communication with the leads 112A/B and a power source 116. The power source 116 can be a battery, such as a rechargeable lithium-ion battery, nickel cadmium battery, or the like. The power source 116, which can be monitored via the battery monitor 134, can be carried in the housing 110 to power the electrodes 112 and computing device 114. Control of the electrodes 112 can be directed by a drive/monitor element 136.

The computing device 114 can include a processor 118, memory 120, 122 and 124, and transceiver circuitry 148. In one embodiment, the processor 118 can be a microprocessor, logic circuit, Application-Specific Integrated Circuit (ASIC) state machine, gate array, controller, or the like. The computing device 114 can generally be configured to control delivery of electrical stimulation according to programmed parameters or a specified treatment protocol. The programmed parameters or specified treatment protocol (e.g., algorithms for therapeutic purposes, etc.) can be stored in the memory 120, 122 and 124 for specific implementation by a control register 132. A clock/calendar element 130 can maintain system timing for the computing device 114. In one embodiment, an alarm drive 128 can be configured to activate one or more notification, alert or alarm features, such as an illuminated, auditory or vibratory alarm 129. In some embodiments, the processor 118 can be configured to receive input from the drive/monitor element 136 and sensor 108 (e.g., accelerometer, microphone, etc. via an optional signal filter 109), which can be configured to monitor for user interaction/user defined commands, motion, heart rate, blood oxygen levels, respiration, perspiration, posture, sound, electromyographic signals, and the like. Accordingly, in some embodiments, the implantable neural stimulation device 102 can detect and use body movement to modify a programmed electrical stimulation regimen, through various combinations of increasing/decreasing intensity, pausing a regularly scheduled therapy session, or initiating a therapy session outside of a regularly scheduled treatment session to meet patient needs and to improve patient outcomes.

The transceiver circuitry 126 can be configured to receive information from and transmit information to one or more physiological sensors 108, external programmer 104, and server 106. The neurostimulator device 102 can be configured to receive programmed parameters and other updates from the external programmer 104, which can communicate with the neurostimulator device 102 through well-known techniques such as wireless telemetry, Bluetooth, or one or more proprietary communication schemes (e.g., Tel-M, Tel-C, etc.). In some embodiments, the external programmer 104 can be configured for exclusive communication with one or more implantable devices 102. In other embodiments, the external programmer 104 can be any computing platform, such as a mobile phone, tablet or personal computer. In some embodiments, the neurostimulator device 102 and external programmer 104 can further be in communication with a cloud-based server 106. The server 106 can be configured to receive, store and transmit information, such as program parameters, treatment protocols, treatment libraries, and patient information, as well as to receive and store data recorded by the neurostimulator device 102.

In embodiments, various notifications, alerts and alarms regarding modification for electrical stimulation therapy, as well as instructional and training programs for receiving and establishing user defined commands for modification of electrical stimulation therapy can be presented by the programmer 104. In one embodiment, the programmer 104 or components thereof can comprise or include various modules or engines, each of which is constructed, programmed, configured, or otherwise adapted to autonomously carry out a function or set of functions. It should also be noted that that stimulator can operate independently of a programmer, such that once a set of instructions is programmed, the neurostimulation device can operate without the need of the programmer, indefinitely. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations, and should generally not be limited to any particular implementation exemplified herein, unless such limitations are expressly called out. In addition, an engine can itself be composed of more than one sub-engine, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

In some embodiments, the programmer 104 can include a processor 140, memory 142, a control engine 144, a communications engine 146, and a power source 148. Processor 140 can include fixed function circuitry and/or programmable processing circuitry. Processor 140 can include any one or more of a microprocessor, a controller, a DSP, an ASIC, an FPGA, or equivalent discrete or analog logic circuitry. In some examples, the processor 140 can include multiple components, such as any combination of one or more microprocessors, one or more controllers, one or more DSPs, one or more ASICs, or one or more FPGAs, as well as other discrete or integrated logic circuitry. The functions attributed to processor 140 herein may be embodied as software, firmware, hardware or any combination thereof.

The memory 142 can include computer-readable instructions that, when executed by processor 140 direct the control engine 144 to perform various functions. Memory 142 can include volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Control engine 144 can include instructions to control the components of the programmer 104 and instructions to selectively control the implantable neural stimulation device 102.

The communications engine 146 can include any suitable hardware, firmware, software, or any combination thereof for communicating with other components of the medical device 102 and/or external devices. Under the control of processor 140, the communication engine 146 can receive downlink telemetry from, as well as send uplink telemetry to one or more external devices (e.g., the implantable medical device 102, etc.) using an internal or external antenna. In addition, communication engine 146 can facilitate communication with a networked computing device and/or a computer network 106. For example, communications engine 146 can receive updates to instructions for control engine 144 from one or more external devices. In another example, communications engine 146 can transmit data regarding the state of system 100 to one or more one or more external devices.

Power source 148 is configured to deliver operating power to the components of the programmer 104. Power source 148 can include a battery and a power generation circuit to produce the operating power. In some examples, the battery is rechargeable to allow extended operation. Power source 148 can include any one or more of a plurality of different battery types. In some embodiments, the programmer 104 can further include an external power supply port. In other embodiments, the neurostimulation device can be recharged wirelessly.

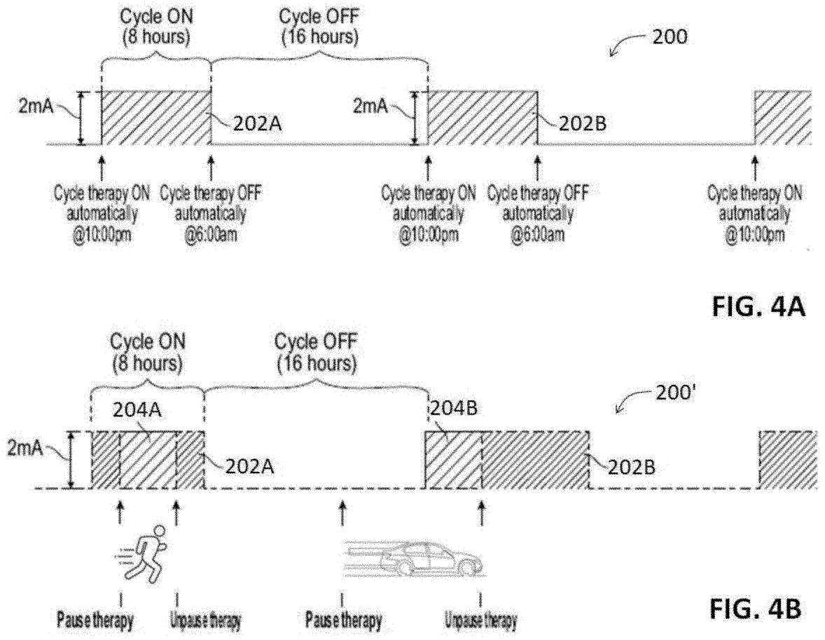

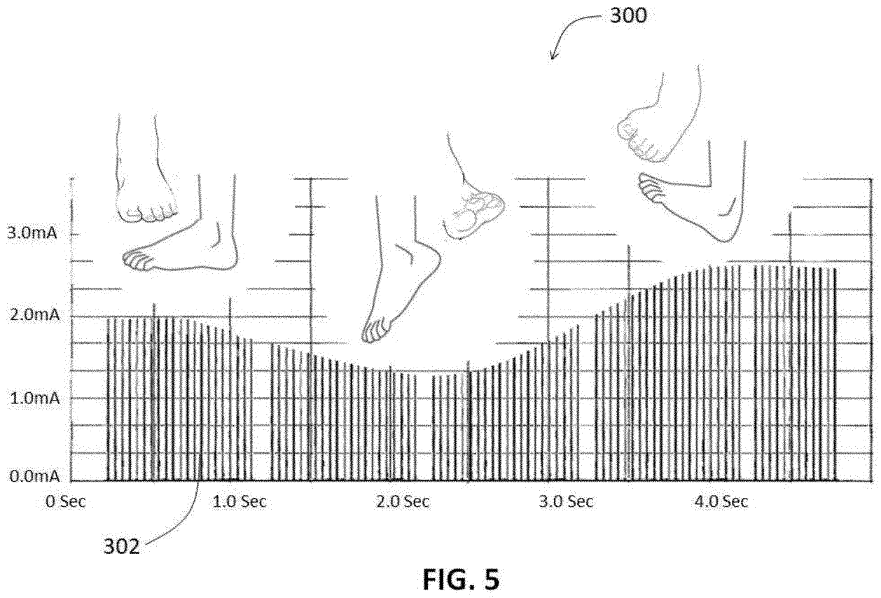

With reference to FIG. 4A a graphical representation of a partial therapeutic regimen 200 is depicted in accordance with an embodiment of the disclosure. According to the partial therapeutic regimen 200, a first dose of electrical neurostimulation 202A is configured to automatically commence at 10 PM for a duration of eight hours with an amplitude of 2 mA. Thereafter, no electrical neurostimulation is scheduled to take place for a period of 16 hours. A subsequent dose of electrical neurostimulation 202B is automatically set to commence at 10 PM within the following 24-hr period for the same duration and amplitude. The on/off timing, total cycle on, total cycle off can be modified programmatically. Additionally, as depicted in FIG. 5, for enhanced therapeutic effect, the on cycle can be ramped up gradually, and the off cycle can be ramped off gradually.

In some embodiments, the disclosed systems and methods can be configured to enable automatically pause or discontinue therapy, thereby enabling patients to temporarily discontinue neurostimulation therapy in a manner that does not interfere with acts of daily living or present other safety issues. For example, with reference to FIG. 4B a graphical representation of a pause 204A within a therapeutic regimen 202A is depicted in accordance with an embodiment of the disclosure. In particular, FIG. 4B depicts an automatic pausing of the electrical neural stimulation at 12 AM (during the first dose of electrical neurostimulation 202A) in response to sensed physical activity, and un-pausing the electrical neural stimulation at 4 AM, thereby enabling the remaining first dose of electrical neural stimulation 202A to resume.

A second pause 204B is initiated at 4 PM and removed or un-paused at 2 AM, during the second dose of electrical neurostimulation 202B in response to sensed motion (e.g., driving). Accordingly, in some embodiments, the therapy can be programmed for a defined duration within a greater therapeutic window (e.g., 8-hrs of therapy within a 24-hr window), regardless of the number of interruptions. In some embodiments, through the use of an internal clock 130, the neurostimulator device 102, is able to maintain the prescribed therapy regimen while enabling the patient to “override” the regimen by selectively pausing therapy delivery (e.g., to avoid pain, discomfort, or when undertaking tasks where no neurostimulation therapy is desired or safety may be of concern).

With additional reference to FIG. 5 a graphical representation of a therapy regimen 300, in which an amplitude of the individual electrical pulses 302 are plotted over a period of time, is depicted in accordance with an embodiment of the disclosure. As depicted, initially, the therapy regimen 300 can be programmed to deliver electrical pulses with an amplitude of about 2 mA. As a motion or position of the user's lower leg is sensed by sensor 108 (or a signal from a targeted nerve is sensed directly via an EMG sensor 108) the amplitude of the individual electrical pulses 302 can be modified. For example, where it is determined that a distance between the electrodes 112 of the implanted device 102 and the targeted nerve has decreased (e.g., as a result of eversion, plantar flexion, etc), the amplitude of the individual pulses 302 can be reduced. Conversely, where it is detected that a distance between the electrodes 112 and the targeted nerve has increased (e.g., as a result of inversion, dorsiflexion, etc), the amplitude of the individual pulses 302 can be increased, thereby maintaining a desired degree of electrical stimulation of the targeted nerve throughout a normal range of motion.

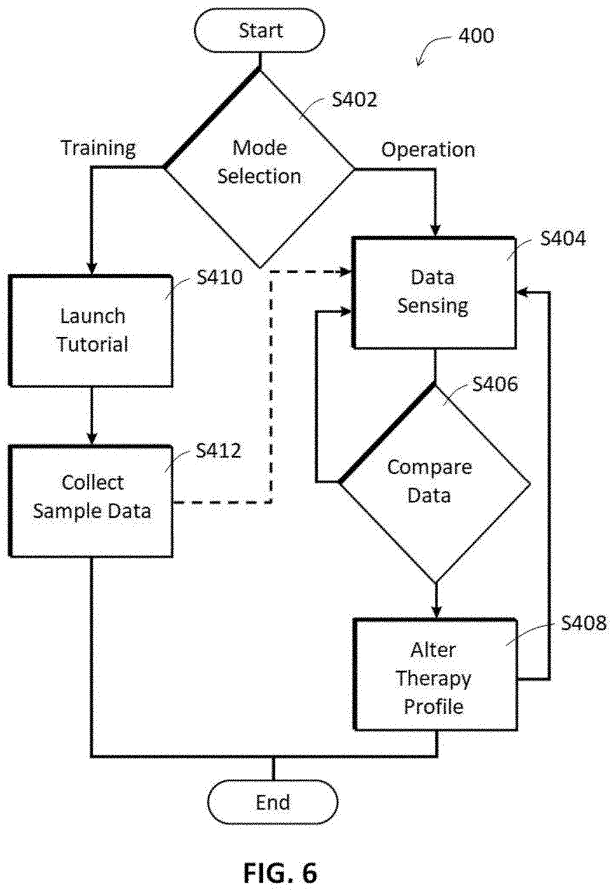

With reference to FIG. 6, a flowchart depicting a method 400 of detecting unintentional and intentional body signals to control and modify electrical stimulation therapy, is depicted in accordance with an embodiment of the disclosure. According to such embodiments, a scheduled of prescribed therapy regimen can be automatically or manually altered or modified (e.g., via one or more user defined commands, sensed information, or combination thereof) thereby improving safety (e.g., not providing electrical stimulation while operating heavy machinery, avoiding patient discomfort, etc.) with ease (e.g., requiring no external communications or complex instructions), while optimizing therapy for improved results.

According to such a method, at S402, a user can select the mode of operation (e.g., training operation, treatment programming, etc.), via programmer 104, which can be communicated to the neurostimulator device 102. If the operation mode is selected, the therapy regimen details can be executed as needed by the drive/monitor element 136. During operation, data can be sensed at S404. Such data can include, but is not limited to, user interaction/user defined commands, motion, heart rate, blood oxygen levels, respiration, perspiration, posture, sound, electromyographic signals, and the like. In some embodiments, the data gathered at S404 can be acquired solely from the implantable device 102, thereby reducing the need for the implantable device to communicate with external devices. In other embodiments, information from one or more sensors outside of the body of the patient (e.g., a wearable sensor, data from a mobile computing device, etc.) can be utilized.

At S406, the data sensed at S404 can be compared to an established standard or model for determination of whether the prescribed therapy profile should be altered. The established standard or model can include, but is not limited to, established user defined commands, anatomical data representing typical movements of a targeted nerve over a range of motion, movement, sound or vibration indicating operation of heavy machinery, sleeping or awake states, and the like. If it is determined that the data sensed at S404 matches the established standard or model, at S408 the therapeutic profile can be altered, for example by reducing the intensity (e.g., amplitude, frequency, duration, etc.) of the electrical stimulation or by pausing the electrical stimulation altogether. Thereafter, the method 400 can continue to monitor for additional data at S404, such that electrical stimulation can resume the initial prescribed therapy regimen upon removal of the match between the established standard or model and the sensed data, or a subsequent user defined command is received.

If at 402, user selects the training mode of operation, at S410 a tutorial can be launched (e.g., via programmer 104). In embodiments, the tutorial can walk the user through a sequence of steps to receive user defined commands or record other inputs indicative of criteria establishing a desire to modify a prescribed therapy regimen. At S412, a variety of data samples can be collected and coalesced into one or more established standards or models for use at S406. In some embodiments, the method 200 can then proceed directly to S404 for operation.

It should be understood that the individual steps used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An implantable neurostimulation device, comprising:
circuitry for generating an electrical stimulation therapy;
one or more electrodes configured to deliver the electrical stimulation therapy to a targeted nerve of a patient; and
at least one sensor configured to sense data used by the circuitry to infer a distance between the one or more electrodes and the targeted nerve, wherein the circuitry is configured to modify the electrical stimulation therapy based on the inferred distance between the one or more electrodes in the targeted nerve.

2. The implantable neurostimulation device of claim 1, wherein the implantable neurostimulation device is adapted for tibial nerve stimulation.

3. The implantable neurostimulation device of claim 2, wherein the at least one sensor is configured to sense at least one of inversion, eversion, dorsiflexion, or plantar flexion in an ankle of the patient.

4. The implantable neurostimulation system of claim 1, wherein the implantable neurostimulation device is configured to at least one of selectively increase or decrease an amplitude of the electrical stimulation therapy based on the inferred distance between the one or more electrodes in the targeted nerve.

5. The implantable neurostimulation system of claim 1, wherein the amplitude of the electrical stimulation therapy is variable in a range of between about 0.1 mA and about 25 mA.

6. The implantable neurostimulation device of claim 1, wherein the one or more sensor is at least one of an accelerometer or electromyographic sensor.

7. The implantable neurostimulation device of claim 1, wherein the implantable neurostimulation device is configured to communicate with at least one of a mobile computing device, a desktop computer, smart watch, or a dedicated implantable neurostimulation device programmer.

8. The implantable neurostimulation device of claim 1, wherein the implantable neurostimulation device is configured to selectively pause the electrical stimulation therapy in response to a user defined tactile command.

9. The implantable neurostimulation device of claim 1, wherein the at least one sensor is further configured to sense data used by the circuitry to infer receipt of a user defined tactile command.

10. The implantable neurostimulation device of claim 9, wherein the implantable neurostimulation device is configured to at least one of pause the electrical stimulation therapy, skip an electrical stimulation therapy session, or change an amplitude of the electrical stimulation therapy in response to an inferred receipt of the user defined tactile command.

11. The implantable neurostimulation device of claim 1, wherein the at least one sensor is further configured to sense data used by the circuitry to infer operation of at least one of a vehicle or heavy machinery.

12. The implantable neurostimulation device of claim 10, wherein the implantable neural stimulation device is configured to selectively pause the electrical stimulation therapy in response to the inferred operation of at least one of a vehicle or heavy machinery.

13. An implantable neurostimulation system, comprising:
an implantable neurostimulation device configured to deliver an electrical stimulation therapy to a targeted nerve of a patient via one or more electrodes; and
an external sensor configured to sense patient activity inferring a change in distance between the one or more electrodes and the targeted nerve, wherein the implantable neurostimulation device is configured to modify the electrical stimulation therapy based on the inferred change in distance between the one or more electrodes in the targeted nerve.

14. The implantable neurostimulation device of claim 13, wherein the implantable neurostimulation device is adapted for tibial nerve stimulation.

15. The implantable neurostimulation device of claim 13, wherein the external sensor is at least one of a heart rate monitor, pulse oximeter, respiratory sensor, perspiration sensor, posture orientation sensor, motion sensor, accelerometer, or microphone.

16. The implantable neurostimulation device of claim 13, wherein the at least one signal correlates to patient operation of at least one of an automobile or heavy machinery.

17. The implantable neurostimulation device of claim 16, wherein the implantable neurostimulation device is configured to selectively pause the electrical stimulation therapy.

18. An implantable neurostimulation system, comprising:
an implantable neurostimulation device configured to deliver an electrical stimulation therapy to a targeted nerve of a patient via one or more electrodes, the implantable neural stimulation device including at least one sensor configured to sense patient activity inferring a change in a distance between the one or more electrodes and the targeted nerve; and
an external programmer configured to wirelessly communicate with the implantable neurostimulation device.

19. The implantable neurostimulation system of claim 18, wherein the at least one sensor is further configured to sense data used by the circuitry to infer receipt of a user defined tactile command.

20. The implantable neurostimulation system of claim 19, wherein the implantable neurostimulation device is configured to at least one of pause the electrical stimulation therapy, skip an electrical stimulation therapy session, or change an amplitude of the electrical stimulation therapy in response to an inferred receipt of the user defined tactile command.
